(19) United States
(12) Patent Application Publication
(10) Pub. No.: US 2024/0335238 A1
(43) Pub. Date: Oct. 10, 2024

(54) SURGICAL SYSTEM, CONTROL METHOD, AND PROGRAM

(71) Applicant: SONY GROUP CORPORATION, TOKYO (JP)

(72) Inventors: HIROMITSU MATSUURA, TOKYO (JP); SHINJI KATSUKI, TOKYO (JP); MOTOAKI KOBAYASHI, TOKYO (JP)

(21) Appl. No.: 18/293,382

(22) PCT Filed: Mar. 7, 2022
(86) PCT No.: PCT/JP2022/009610

§ 371 Date: Jan. 30, 2024

(30) Foreign Application Priority Data
Aug. 26, 2021 (JP) 2021-138108

(51) Int. Cl. A61B 34/20; G06T 7/11

(52) U.S. Cl. CPC A61B 34/20; G06T 7/11

(57) ABSTRACT

The present technology relates to a surgical system, a control method, and a program that enable a region-of-interest of an operator to be appropriately set. The surgical system according to one aspect of the present technology performs segmentation of an image captured by a camera and sets a segmentation region in which each target is displayed, acquires a region-of-interest candidate that is a region to be a candidate for a region-of-interest of an operator, and sets the region-of-interest on the basis of a relationship between the segmentation region and the region-of-interest candidate. The present technology can be applied to a surgical system using an endoscope.

### TECHNICAL FIELD

The present technology relates to a surgical system, a control method, and a program, and more particularly, to a surgical system, a control method, and a program that enable a region-of-interest of an operator to be appropriately set.

### BACKGROUND ART

In a surgical system using an endoscope or the like, sterilization measures of an operator are required. Therefore, various techniques have been proposed in which a device such as an endoscope can be operated in a non-contact manner.

Patent Document 1 discloses a technique of controlling a focus of a camera by non-contact input using a voice, a gesture, a line-of-sight, or the like of an operator.

Furthermore, Patent Document 2 discloses a technique of controlling focus and exposure of a camera by performing image segmentation.

### CITATION LIST

### Patent Document

Patent Document 1: Japanese Patent Application Laid-Open No. 2017-070636
Patent Document 2: WO 2018/003503

### SUMMARY OF THE INVENTION

### Problems to be Solved by the Invention

Generally, non-contact input is more likely to be misrecognized than contact input. In some cases, misrecognition of the input causes malfunction of the surgical system.

For example, in a case where the line-of-sight is used as an input in a non-contact manner, in some cases, it is erroneously recognized that the operator is focusing on an organ next to an operation target organ, and control is performed to focus the endoscope on the organ next to the operation target organ. Because the line-of-sight of the operator during the surgery is often directed not to the center but to the end of the operation target organ, there are cases where the operator is erroneously recognized as focusing on the organ next to the operation target organ.

The present technology has been made in view of such a situation, and enables a region-of-interest of an operator to be appropriately set.

### Solutions to Problems

A surgical system according to one aspect of the present technology includes: an image processing unit that performs segmentation of an image captured by a camera and sets a segmentation region in which each target is displayed; a region-of-interest candidate acquisition unit that acquires a region-of-interest candidate that is a region to be a candidate for a region-of-interest of an operator; and a control unit that sets the region-of-interest on the basis of a relationship between the segmentation region and the region-of-interest candidate.

According to one aspect of the present technology, the segmentation of the image captured by the camera is performed and the segmentation region in which each target is displayed is set, the region-of-interest candidate that is a region to be a candidate for the region-of-interest of the operator is acquired, and the region-of-interest is set on the basis of a relationship between the segmentation region and the region-of-interest candidate.

### BRIEF DESCRIPTION OF DRAWINGS

FIG. 1 is a diagram illustrating a configuration example of a surgical system to which the present technology is applied.

FIG. 2 is a diagram illustrating an example of an operative field image.

FIG. 3 is a diagram illustrating an example of a region-of-interest candidate and a segmentation region.

FIG. 4 is a diagram illustrating an example of a setting method of a region-of-interest.

FIG. 5 is a block diagram illustrating a configuration example of a control device in FIG. 1.

FIG. 6 is a flowchart for explaining a series of processing in the control device in FIG. 1.

FIG. 7 is a flowchart for explaining processing in the control unit performed in step S3 in FIG. 6.

FIG. 8 is a diagram illustrating an example of the segmentation region.

FIG. 9 is a diagram illustrating an example of coupling of the segmentation regions.

FIG. 10 is a block diagram illustrating a configuration example of hardware of a computer.

### MODE FOR CARRYING OUT THE INVENTION

Hereinafter, modes for carrying out the present technology is described. The description is given in the following order.
1. First embodiment (Example of setting method of region-of-interest)
2. Configuration of control device
3. Operation of control device
4. Second embodiment (Setting of segmentation region)
5. Third embodiment (Measures taken in a case where segmentation region is small)
6. Fourth embodiment (Measures taken in a case where there is error in region-of-interest candidate)
7. Fifth embodiment (Weighting for segmentation region)
8. Sixth embodiment (Division of segmentation region by using Depth information)
9. Seventh embodiment (Coupling of segmentation regions by using Depth information)
10. Eighth embodiment (Division of segmentation region by using SLAM information)
11. Ninth embodiment (Coupling of segmentation regions by using SLAM information)
12. Tenth embodiment (Display of region-of-interest)
13. Eleventh embodiment (Setting change of region-of-interest by speech)
14. Twelfth embodiment (Example of acquisition source of surgical procedure information)
15. Thirteenth embodiment (Setting of region-of-interest corresponding to display magnification)
16. Others

### First Embodiment (Example of Setting Method of Region-of-Interest)

Configuration Example of Surgical System to which Present Technology is Applied

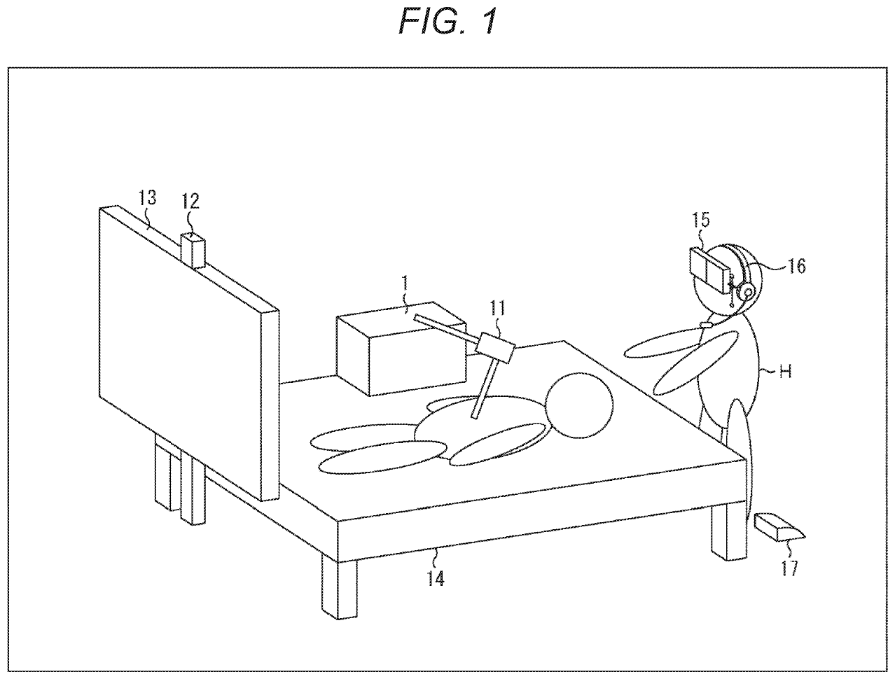

FIG. 1 is a diagram illustrating a configuration example of a surgical system according to an embodiment of the present technology.

The surgical system in FIG. 1 includes a control device 1, a surgical camera 11, a motion recognition camera 12, a display 13, an operating table 14, a line-of-sight recognition device 15, a microphone 16, and a foot switch 17. The surgical system is a system disposed in an operating room or the like and used for treatment such as surgery performed with reference to an image captured by the surgical camera 11. A treatment is performed by an operator H wearing the line-of-sight recognition device 15 and the microphone 16 on the head.

The surgical camera 11 is, for example, a camera used for imaging an operative field in laparoscopic surgery. The surgical camera 11 images an operative field or the like of a patient lying on the operating table 14, and transmits an image obtained as a result to the control device 1 as an operative field image. As the operative field image, a moving image or a still image is captured.

The motion recognition camera 12 is a camera used for recognizing the motion of the operator H. The motion recognition camera 12 is disposed, for example, on the display 13. The motion recognition camera 12 images the operator H, and transmits an image obtained as a result to the control device 1 as an operator image.

The display 13 displays an operative field image and the like under control of the control device 1. The display 13 is installed with a display surface facing the operator H.

The control device 1 receives the operator image transmitted from the motion recognition camera 12 and recognizes a gesture of the operator H.

Furthermore, the control device 1 receives information transmitted from the line-of-sight recognition device 15, and recognizes a viewpoint position on the screen of the display 13. The line-of-sight recognition device 15 transmits information on the line-of-sight of the operator H.

The control device 1 receives a voice transmitted from the microphone 16 and performs voice recognition. The control device 1 receives the signal transmitted from the foot switch 17 and recognizes the content of the operation of the operator H on the foot switch 17.

The control device 1 controls imaging of the surgical camera 11 and display of the display 13 on the basis of the recognized information.

As described above, the control device 1 is a device that controls the surgical system on the basis of input of at least one of the voice, the line-of-sight, the touch, the gesture of the operator H, or the operation of the operator H using the foot switch 17.

The microphone 16 acquires the voice of the operator H and transmits the voice to the control device 1.

The foot switch 17 is disposed at the foot of the operator H. The foot switch 17 transmits, to the control device 1, an operation signal indicating the content of the operation of the operator H performed using the foot.

In the surgical system configured as described above, the operator H lays the patient on the operating table 14, and performs treatment such as surgery while viewing the operative field image and the like displayed on the display 13 via the line-of-sight recognition device 15.

Furthermore, in a case where the imaging condition, the position and the angle of the surgical camera 11, the display of the display 13, or the like is changed, the operator H performs input by voice, the line-of-sight, touching, gesturing, and the foot switch operation. By using the voice, the line-of-sight, the gesturing, and the like, the operator H can perform input for the operation of the surgical camera 11 in a non-contact manner while holding a not-illustrated surgical tool.

Note that any method can be adopted as a recognition method of the line-of-sight of the operator H, a detection method of gesture, and an acquisition method of voice.

In the control device 1 that controls the surgical system having the above configuration, a region-of-interest, which is a region considered to be focused by the operator H, is set for the operative field image, and the driving of the surgical camera 11 is controlled in accordance with the region-of-interest. For example, focus control for focusing on the region-of-interest and exposure control in accordance with brightness of the region-of-interest are performed in accordance with the region-of-interest.

Such a region-of-interest used as a determination area for the focus control and the exposure control is set on the basis of a relationship between a region-of-interest candidate that is a candidate for the region-of-interest and a segmentation region set by performing segmentation of an image.

### Example of Setting Method of Region-of-Interest Region

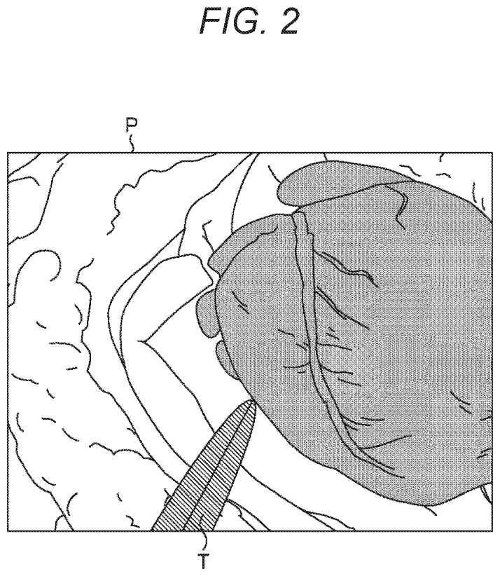

FIG. 2 is a diagram illustrating an example of the operative field image.

Here, a setting method of the region-of-interest of the operator H is described using an operative field image P illustrated in FIG. 2. In the operative field image P, a region on the right side indicated by color is a region in which an operation target organ is displayed. Other organs are displayed around the operation target organ. Furthermore, a portion around the distal end of a surgical tool T is displayed in a region indicated by hatching below the center of the operative field image P.

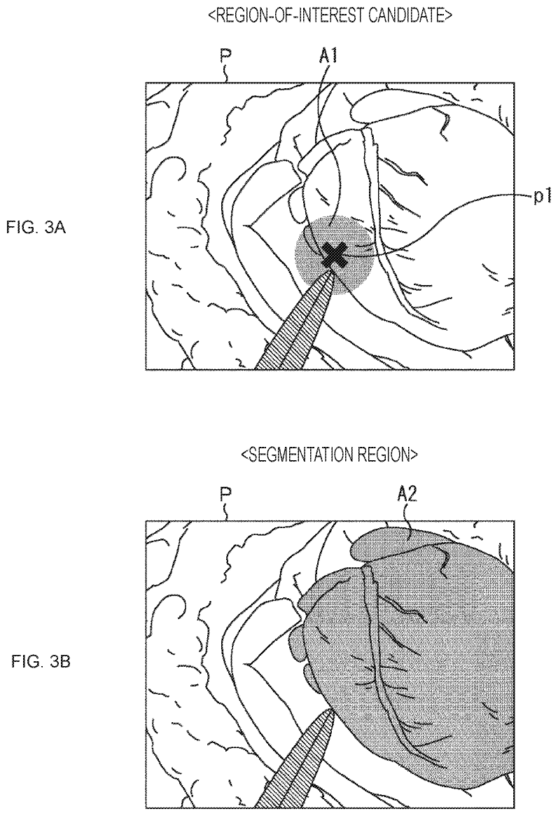

In a case where the operative field image P is captured by the surgical camera 11, in the control device 1, for example, on the basis of the information supplied from the line-of-sight recognition device 15, a region-of-interest candidate A1 is set in a colored manner as illustrated in A of FIG. 3. In A of FIG. 3, a circular range of a certain distance around a viewpoint position p1 is set as the region-of-interest candidate A1. The viewpoint position p1 is a position in the vicinity of an edge of the operation target organ.

Furthermore, in the control device 1, by performing the segmentation on the operative field image P, a region in which the operation target organ is displayed as illustrated with color in B of FIG. 3 is set as a segmentation region A2. For example, in a case where a plurality of organs is displayed in the operative field image P, a plurality of segmentation regions is set by performing the segmentation, and the segmentation region A2 in which the operation target organ is displayed is used for setting the region-of-interest.

The segmentation of the operative field image P is performed by using, for example, an inference model generated in advance by machine learning using an image displaying each organ as learning data. By inputting the operative field image P to the inference model, information regarding the segmentation region in which each organ is displayed is output.

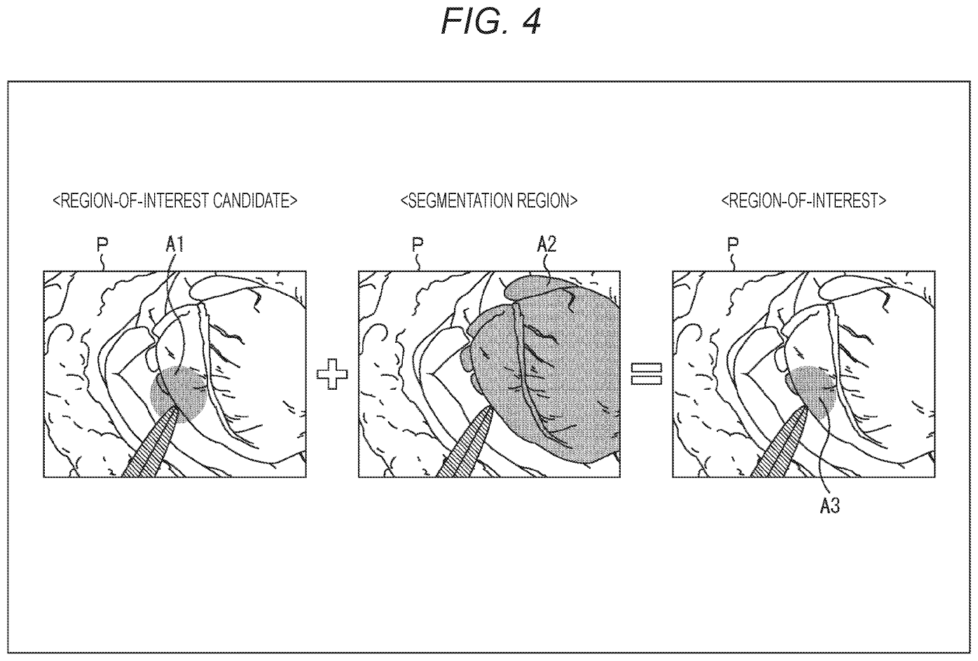

FIG. 4 is a diagram illustrating an example of a setting method of the region-of-interest.

In a case where the region-of-interest candidate A1 and the segmentation region A2 are set as described above, as illustrated in FIG. 4, for example, a common region between the region-of-interest candidate A1 and the segmentation region A2 is set as a region-of-interest A3. In the control device 1, the surgical camera 11 is controlled by focusing on the region-of-interest A3 or adjusting the exposure in accordance with the brightness of the region-of-interest A3.

As described above, in the control device 1, the region-of-interest A3 is set on the basis of the relationship between the region-of-interest candidate A1 and the segmentation region A2.

With this arrangement, a non-object-of-interest displayed at a position close to the viewpoint can be removed from the region-of-interest A3, and a region matching the intention of the operator H can be set as the region-of-interest A3. That is, a region outside the segmentation region A2 among the region-of-interest candidate A1 set on the basis of the viewpoint position is a region in which an organ adjacent to the operation target organ is displayed, and is a region in which the non-object-of-interest is displayed. It can be said that such a region-of-interest A3 set so as to exclude the region in which the non-object-of-interest is displayed is a region matching the intention of the operator H who is focusing on the operation target organ.

Furthermore, by controlling the surgical camera 11 on the basis of the region-of-interest A3, the focus control and the exposure control that match the intention of the operator H can be performed.

Normally, the viewpoint position of the operator H is recognized in a state of constantly shaking. Therefore, in a case where the region-of-interest candidate A1 is set as the region-of-interest only on the basis of the viewpoint position, the surgical camera 11 is controlled in accordance with the shaking of the viewpoint position, and the display of the operative field image changes each time. By setting the region-of-interest A3 by using the segmentation region A2 together with the region-of-interest candidate A1, such a change in display can be suppressed.

The setting of the region-of-interest A3 may be performed on the basis of a degree of importance set at each position in the segmentation region A2, instead of uniformly setting the common region of the region-of-interest candidate A1 and the segmentation region A2 as the region-of-interest A3. In this case, for example, weighting corresponding to the distance from the viewpoint position is performed, and the degree of importance is set for each position in the segmentation region A2. Furthermore, the region-of-interest A3 is set so as to include the position where the degree of importance equal to or more than a threshold value is set. The setting of the region-of-interest A3 using the degree of importance will be described later.

### <Configuration of Control Device>

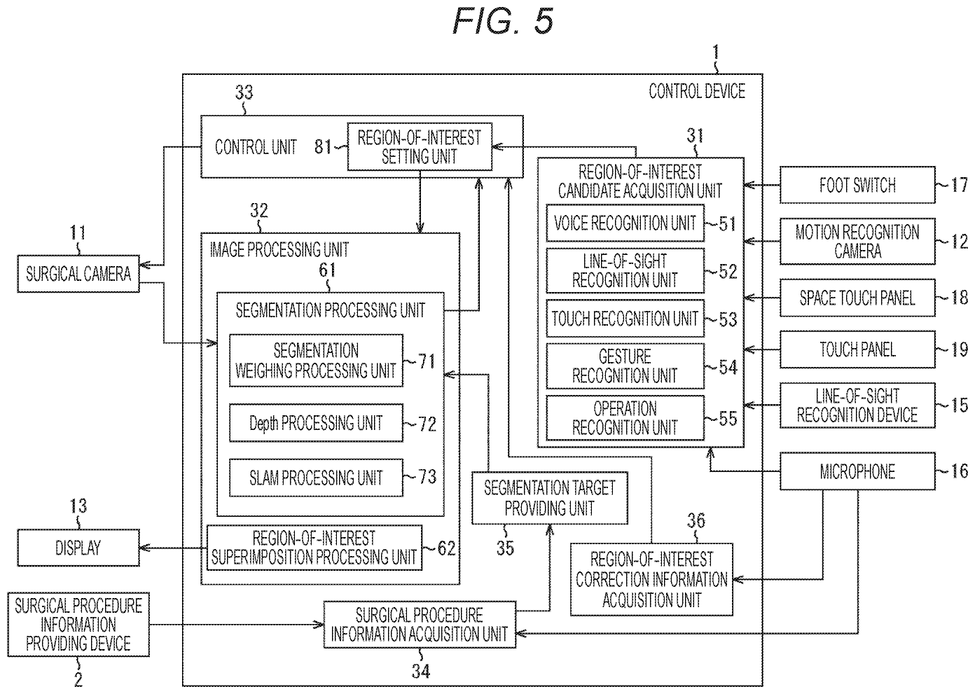

FIG. 5 is a block diagram illustrating a configuration example of the control device 1 in FIG. 1. In the configurations illustrated in FIG. 5, the same configurations as the configurations described with reference to FIG. 1 are denoted by the same reference numerals. Redundant descriptions are omitted as appropriate.

The control device 1 includes a region-of-interest candidate acquisition unit 31, an image processing unit 32, a control unit 33, a surgical procedure information acquisition unit 34, a segmentation target providing unit 35, and a region-of-interest correction information acquisition unit 36. Each functional unit as illustrated in FIG. 5 is realized by executing a predetermined program by a computer constituting the control device 1.

The region-of-interest candidate acquisition unit 31 includes a voice recognition unit 51, a line-of-sight recognition unit 52, a touch recognition unit 53, a gesture recognition unit 54, and an operation recognition unit 55. Information output from each of the input devices of the motion recognition camera 12, the line-of-sight recognition device 15, the microphone 16, the foot switch 17, a space touch panel 18, and a touch panel 19 is input to the region-of-interest candidate acquisition unit 31.

The voice recognition unit 51 performs the voice recognition on the basis of the voice of the operator H supplied from the microphone 16.

The line-of-sight recognition unit 52 recognizes a viewpoint position on the screen of the display 13 on the basis of information on the line-of-sight of the operator H supplied from the line-of-sight recognition device 15.

The touch recognition unit 53 recognizes the content of touch input of the operator H on the basis of operation signals supplied from the space touch panel 18 and the touch panel 19. The space touch panel 18 is an input device that detects input of the operator H performed by using a finger or a hand with respect to a predetermined space. The space touch panel 18 is provided at a predetermined position of the surgical system. The touch panel 19 is provided, for example, in an overlapping manner on the display 13.

The gesture recognition unit 54 recognizes the content of gesture input of the operator H on the basis of an operator image supplied from the motion recognition camera 12.

The operation recognition unit 55 recognizes the content of input of the operator H on the basis of an operation signal supplied from the foot switch 17.

The region-of-interest candidate acquisition unit 31 acquires (sets) the region-of-interest candidate on the basis of the voice recognition result, the viewpoint position, the touch input, the gesture input, and the foot switch input, which are recognition results in each unit. The region-of-interest candidate acquisition unit 31 outputs information on the region-of-interest candidate to the control unit 33.

In this manner, the region-of-interest candidate can be acquired on the basis of information other than the viewpoint position. For example, in a case where a speech such as “near the surgical tool” is made, a region in the vicinity of the distal end of the surgical tool is set as the region-of-interest candidate on the basis of the result of voice recognition.

Instead of setting the region-of-interest candidate on the basis of one recognition result, the region-of-interest candidate may be set on the basis of two or more recognition results. Setting of the region-of-interest candidate can be made on the basis of at least one of the voice recognition result, the viewpoint position, the touch input, the gesture input, and the foot switch input.

The image processing unit 32 includes a segmentation processing unit 61 and a region-of-interest superimposition processing unit 62.

The segmentation processing unit 61 performs segmentation on the operative field image supplied from the surgical camera 11, and outputs information regarding a result of the segmentation to the control unit 33. The information supplied to the control unit 33 includes information of each segmentation region.

The segmentation processing unit 61 includes a segmentation weighting processing unit 71, a Depth processing unit 72, and a SLAM processing unit 73. The function of each of the units included in the segmentation processing unit 61 will be described later. The setting of the region-of-interest is performed by the control unit 33 by appropriately using the information acquired by each of the segmentation weighting processing unit 71, the Depth processing unit 72, and the SLAM processing unit 73.

The region-of-interest superimposition processing unit 62 displays the region-of-interest on the display 13 on the basis of the information supplied from a region-of-interest setting unit 81 of the control unit 33. The region-of-interest is displayed so as to be superimposed on the operative field image.

The control unit 33 includes the region-of-interest setting unit 81. The region-of-interest setting unit 81 sets the region-of-interest on the basis of a relationship between the region-of-interest candidate represented by the information supplied from the region-of-interest candidate acquisition unit 31 and the segmentation region represented by the information supplied from the segmentation processing unit 61 of the image processing unit 32. The region-of-interest setting unit 81 outputs information on the region-of-interest to the image processing unit 32.

Furthermore, the control unit 33 controls driving of the surgical camera 11 on the basis of the region-of-interest.

The surgical procedure information acquisition unit 34 receives and acquires the surgical procedure information supplied from a surgical procedure information providing device 2. The surgical procedure information includes information such as the operation content and an operation target organ. The surgical procedure information acquired by the surgical procedure information acquisition unit 34 is supplied to the segmentation target providing unit 35. The acquisition of the surgical procedure information by the surgical procedure information acquisition unit 34 is appropriately performed on the basis of the voice supplied from the microphone 16.

The segmentation target providing unit 35 specifies a region to be set as a segmentation region on the basis of the surgical procedure information supplied from the surgical procedure information acquisition unit 34 and provides the region to the segmentation processing unit 61 of the image processing unit 32. For example, an operation target organ is specified on the basis of the surgical procedure information, and information indicating that the operation target organ is set as a segmentation region is provided to the segmentation processing unit 61.

The region-of-interest correction information acquisition unit 36 generates correction information that is information instructing correction (change) of the region-of-interest on the basis of the voice supplied from the microphone 16, and outputs the correction information to the control unit 33. For example, in a case where the operator H makes a speech with the content requesting to change the region-of-interest, the correction information is generated. The region-of-interest is appropriately changed on the basis of the correction information generated by the region-of-interest correction information acquisition unit 36. The correction of the region-of-interest may be instructed on the basis of a non-contact input other than the voice input.

### <Operation of Control Device>

Here, movement of the control device 1 having the above configuration is described.

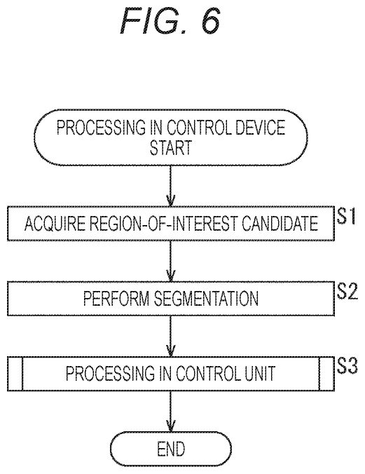

First, with reference to a flowchart in FIG. 6, a series of processing in the control device 1 is described.

In step S1, the region-of-interest candidate acquisition unit 31 acquires the region-of-interest candidate of the operator H.

In step S2, the image processing unit 32 performs segmentation of the operative field image and sets a region in which the operation target organ is displayed as a segmentation region.

In step S3, processing in the control unit 33 is performed.

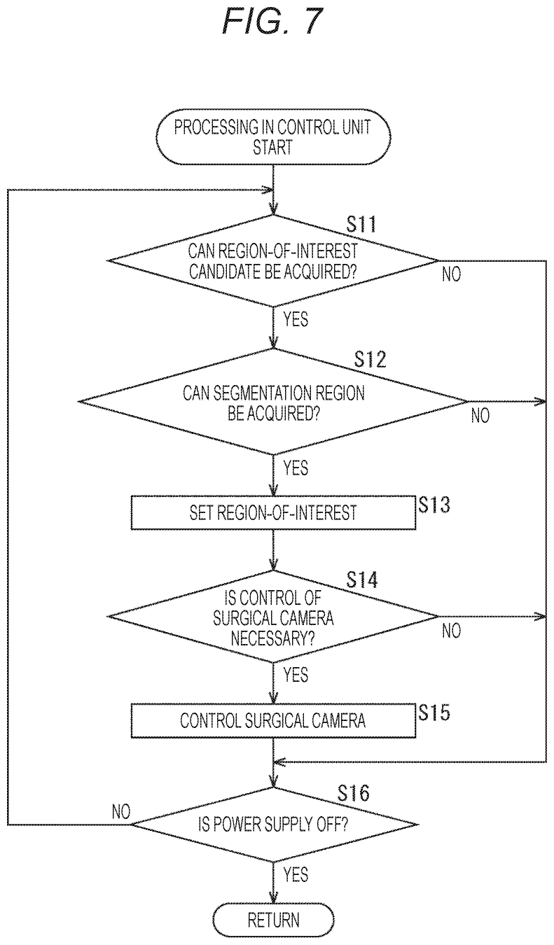

Next, processing in the control unit performed in step S3 in FIG. 6 is described with reference to a flowchart in FIG. 7.

In step S11, the control unit 33 determines whether or not the region-of-interest candidate can be acquired. For example, in a case where the information regarding the recognition result of the viewpoint position of the operator H is included in the information supplied from the region-of-interest candidate acquisition unit 31, it is determined that the region-of-interest candidate can be acquired.

In a case where it is determined in step S11 that the region-of-interest candidate can be acquired, in step S12, the control unit 33 determines whether or not the segmentation region can be acquired. For example, in a case where the segmentation of the operative field image is performed by the segmentation processing unit 61 and the information on the segmentation region is included in the information supplied from the segmentation processing unit 61, it is determined that the segmentation region can be acquired.

In a case where it is determined in step S12 that the segmentation region can be acquired, in step S13, the control unit 33 sets the region-of-interest on the basis of a relationship between the region-of-interest candidate and the segmentation region. As described above, for example, a common region between the region-of-interest candidate and the segmentation region is set as the region-of-interest.

In step S14, the control unit 33 determines whether or not control of the surgical camera 11 is necessary. For example, in a case where there is a change in the region-of-interest, it is determined that the control of the surgical camera 11 is necessary.

In a case where it is determined in step S14 that the control of the surgical camera 11 is necessary, in step S15, the control unit 33 controls at least one of the focus and the exposure of the surgical camera 11 according to the situation of the region-of-interest.

After the driving of the surgical camera 11 is controlled in step S15, the processing proceeds to step S16. In a case where it is determined in step S11 that the region-of-interest candidate cannot be acquired, in a case where it is determined in step S12 that the segmentation region cannot be acquired, or in a case where it is determined in step S14 that the control of the surgical camera 11 is not necessary, the processing similarly proceeds to step S16.

In step S16, the control unit 33 determines whether or not to turn off the power supply of the control device 1.

In a case where it is determined in step S16 that the power supply of the control device 1 is not to be turned off, the processing returns to step S11, and the above processing is repeated.

In a case where it is determined in step S16 that the power supply of the control device 1 is to be turned off, the processing returns to step S3 in FIG. 6, and the processing in the control device 1 is ended.

Through the above processing, the control device 1 can appropriately set the region-of-interest on the basis of the relationship between the region-of-interest candidate and the segmentation region. Furthermore, the control device 1 can appropriately control the surgical camera 11 on the basis of the region-of-interest set so as to match the intention of the operator H.

### Second Embodiment (Setting of Segmentation Region)

Instead of setting one segmentation region in the entire region in which the operation target organ is displayed, a plurality of segmentation regions may be set.

For example, in a case where the operation target organ is the large intestine, each region in which a site such as the transverse colon or the upper rectum is displayed, and each region having a narrower width such as the mesentery or the blood vessel is displayed are set as the segmentation region.

In this case, for example, on the basis of the surgical procedure information supplied from the surgical procedure information acquisition unit 34, the segmentation target providing unit 35 in FIG. 5 sets a granularity of a region to be set as the segmentation region. On the basis of the granularity set by the segmentation target providing unit 35, the segmentation processing unit 61 sets a region in which a portion of one operation target organ is displayed as the segmentation region.

With this arrangement, a narrower region-of-interest can be set.

In the operation target organ, a region in which a portion with a tumor is displayed and a region in which a portion without a tumor is displayed may be set as different segmentation regions.

### Third Embodiment (Measures Taken in a Case where Segmentation Region is Small)

A common region between one region-of-interest candidate and each of a plurality of segmentation regions may be set as the region-of-interest.

In this case, the segmentation processing unit 61 sets the plurality of segmentation regions in the operative field image. The region-of-interest setting unit 81 sets a common region between the region-of-interest candidate and each of the segmentation regions as the region-of-interest.

With this arrangement, even in a case where one segmentation region is narrow, a region having a certain size as the region-of-interest serving as a reference of autofocus control and exposure control can be secured.

### Fourth Embodiment (Measures Taken in a Case where there is Error in Region-of-Interest Candidate)

In a case where it is difficult to set the region-of-interest because there is an error in the region-of-interest candidate at each timing due to the line-of-sight of the operator H moving, the region-of-interest may be set also on the basis of the positional relationship between the surgical tool and the operation target organ.

In this case, for example, the surgical process is determined on the basis of the positional relationship between the surgical tool and the operation target organ with reference to the surgical procedure information. In surgery using an endoscope, because a location to be treated is standardized according to a surgical procedure, the surgical process can be determined on the basis of the positional relationship between the surgical tool and the organ.

The segmentation weighting processing unit 71 specifies a separating portion and a cutting portion of the operation target organ, and sets a high degree of importance to, for example, a portion in which the organ sandwiched by a pair of forceps is displayed. The region-of-interest setting unit 81 sets the region-of-interest on the basis of the degree of importance so as to include the portion in which the organ sandwiched by the forceps is displayed. For example, the region-of-interest is set so as to include a portion where the degree of importance equal to or more than a threshold value is set.

With this arrangement, even in a case where there is an error in the region-of-interest candidate at each timing, the region-of-interest setting unit 81 can appropriately set the region-of-interest.

### Fifth Embodiment (Weighting for Segmentation Region)

Each of the portions of the segmentation region may be weighted such that a region where a tumor portion is displayed is preferentially included in the region-of-interest.

In this case, for example, the segmentation weighting processing unit 71 specifies a region in which a tumor portion of an operation target organ is displayed on the basis of the surgical procedure information acquired by the surgical procedure information acquisition unit 34, and sets a high degree of importance to the specified region. Furthermore, on the basis of the degree of importance set for each region, the region-of-interest setting unit 81 sets a region including the region in which the tumor portion is displayed as the region-of-interest.

With this arrangement, the focus control and the exposure control matching the intention of the operator H.

Each region may be weighted so as to include a region with high contrast such as a region in which the surgical tool is displayed in the region-of-interest. By performing the focus control on the basis of the region-of-interest including the region with high contrast, the focus performance can be improved.

### Sixth Embodiment (Division of Segmentation Region by Using Depth Information)

The segmentation region in which the operation target organ is displayed may be divided into a plurality of segmentation regions on the basis of Depth information of the operation target organ.

In this case, the Depth processing unit 72 performs Depth estimation using the operative field image captured by the surgical camera 11, and acquires Depth information indicating the distance to each portion appearing in the operative field image. The Depth estimation performed by the Depth processing unit 72 is so-called monocular Depth estimation.

Furthermore, in a case where the operation target organ is an object having a deep depth of field necessary for focusing (in a case where the operation target organ has a wide width in the depth direction), the segmentation processing unit 61 divides the entire region in which the operation target organ is displayed into a plurality of segmentation regions.

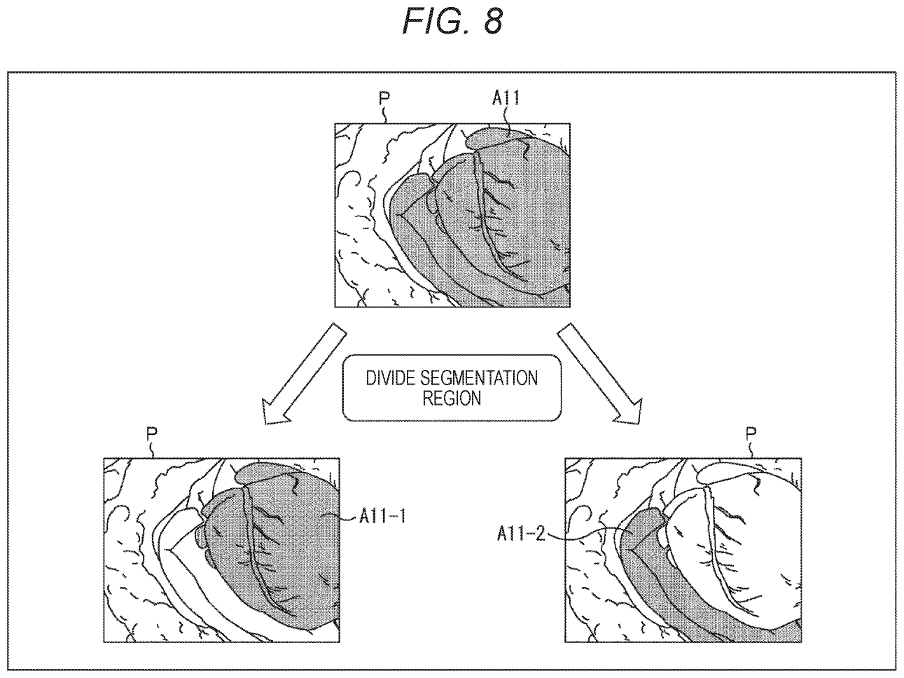

FIG. 8 is a diagram illustrating an example of dividing the segmentation region.

In the example in FIG. 8, an operation target organ is displayed in the operative field image P, and a segmentation region A11 is set. In a case where the entire segmentation region A11 is difficult to be focused, the segmentation region A11 is divided into a segmentation region A11-1 and a segmentation region A11-2 as indicated by arrows in FIG. 8. For example, the segmentation region is divided on the basis of the Depth information such that the distance to each position in the region falls within a certain distance.

With this arrangement, the focus can be appropriately adjusted by using either the segmentation region A11-1 or the segmentation region A11-2, which are at about the same distance, for setting the region-of-interest.

In a surgical system using an endoscope, because a distance to an object to be a subject is short, an achievable depth of field becomes shallow. Furthermore, a pixel pitch of an image sensor used for the endoscope is narrowed due to enhanced resolution, and accordingly, the achievable depth of field also becomes shallow. As described above, by dividing the segmentation region such that the distance to each position in the region falls within a certain distance, the focus can be appropriately adjusted even in a case where the region-of-interest is set in any region in the segmentation region.

Furthermore, in order not to damage other organs, treatment such as incision or excision is performed in a state where the operation target organ is lifted with the forceps. In this case, the depth of field required to focus on the entire organ becomes deep, but as described above, because the segmentation region is divided such that the distance to each position in the region falls within a certain distance, the focus can be appropriately controlled.

### Seventh Embodiment (Coupling of Segmentation Regions by Using Depth Information)

The plurality of segmentation regions in which the operation target organ is displayed may be coupled into one segmentation region on the basis of Depth information of the operation target organ.

In this case, the Depth processing unit 72 performs Depth estimation using the operative field image captured by the surgical camera 11, and acquires Depth information indicating the distance to each portion appearing in the operative field image.

Furthermore, in a case where the operation target organ is an object having a shallow depth of field necessary for focusing (in a case where the operation target organ has a narrow width in the depth direction), the segmentation processing unit 61 couples the plurality of regions in which the operation target organ is displayed into one segmentation region.

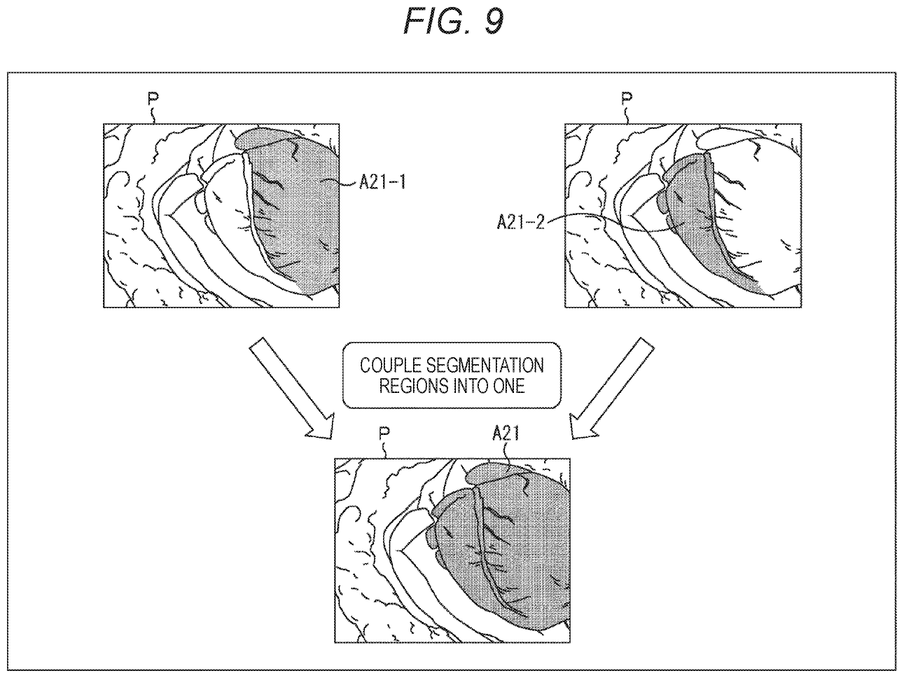

FIG. 9 is a view illustrating an example of coupling of the segmentation regions.

In the example in FIG. 9, an operation target organ is displayed in the operative field image P, and a segmentation region A21-1 and a segmentation region A21-2 are set. In a case where the distance to each of portions, which are a portion displayed in the segmentation region A21-1 and a portion displayed in the segmentation region A21-2, is within a certain range, the segmentation region A21-1 and the segmentation region A21-2 are coupled to one segmentation region A21 as indicated by arrows in FIG. 9.

By using the segmentation region A21, a wide region is set as the region-of-interest serving as a reference for focusing. With this arrangement, the operative field image in a state in which the entire organ displayed in the wide region is in focus.

### Eighth Embodiment (Division of Segmentation Region by Using SLAM Information)

The SLAM information can be used to divide the segmentation region.

In this case, the SLAM processing unit 73 performs the SLAM processing by using the operative field image captured by the surgical camera 11. The segmentation processing unit 61 specifies the distance to each portion displayed in the operative field image on the basis of the SLAM information indicating a result of the SLAM processing, and divides the segmentation region as described with reference to FIG. 8.

Also with this arrangement, the focus of each of the plurality of segmentation regions can be appropriately adjusted.

### Ninth Embodiment (Coupling of Segmentation Regions by Using SLAM Information)

The SLAM information can be used to couple the segmentation regions.

In this case, the SLAM processing unit 73 performs the SLAM processing by using the operative field image captured by the surgical camera 11. The segmentation processing unit 61 specifies the distance to each portion displayed in the operative field image on the basis of the SLAM information indicating a result of the SLAM processing, and couples the segmentation regions as described with reference to FIG. 9.

Also with this arrangement, the operative field image can be captured in a state in which the focus of the entire organ displayed in the wide region is focused.

### Tenth Embodiment (Display of Region-of-Interest)

At the time of controlling at least one of the focus and the exposure of the surgical camera 11, the information regarding the region-of-interest may be fed back to the operator H.

In this case, on the basis of information supplied from the region-of-interest setting unit 81, the region-of-interest superimposition processing unit 62 displays, on the display 13, information representing which region is set as the region-of-interest. For example, an image of a predetermined color is displayed in a superimposed manner on the operative field image, and the region-of-interest is presented to the operator H.

By the region-of-interest being displayed on the display 13, the operator H can appropriately grasp the behavior of the surgical system.

### Eleventh Embodiment (Setting Change of Region-of-Interest by Speech)

The setting of the region-of-interest may be changed in response to the speech of the operator H made after the presentation of the information regarding the region-of-interest.

In this case, the region-of-interest correction information acquisition unit 36 generates correction information that is information instructing correction of the region-of-interest on the basis of the voice supplied from the microphone 16. The correction information is generated in response to a speech made, such as “slightly front”, “slightly behind”, and “wrong”. The region-of-interest setting unit 81 changes the region-of-interest on the basis of the correction information generated by the region-of-interest correction information acquisition unit 36, and controls the surgical camera 11 according to the changed region-of-interest.

With this arrangement, even in a case where the region-of-interest is set in a form not matching the intention of the operator H, the region-of-interest can be appropriately corrected.

### Twelfth Embodiment (Example of Acquisition Source of Surgical Procedure Information)

Although the surgical procedure information is acquired from the surgical procedure information providing device 2 constituting a hospital information system (HIS), the surgical procedure information may be acquired on the basis of the speech at the time of timeout. The timeout is a time for confirming the name of the patient, the surgical procedure, and the surgical site. For example, a time for the timeout is secured before the start of surgery or the like.

In this case, the surgical procedure information acquisition unit 34 recognizes a speech at the time of timeout detected by the microphone 16, and generates the surgical procedure information by specifying the name of the patient, the surgical procedure, and the surgical site. On the basis of the surgical procedure information generated by the surgical procedure information acquisition unit 34, setting of the degree of importance and the like are performed. That is, the surgical procedure information acquisition unit 34 can acquire the surgical procedure information on the basis of at least one of the information transmitted from the cooperative HIS and the recognition result of the speech of the operator H or the like before the start of the surgery.

With this arrangement, the number of options of the acquisition source of the surgical procedure information can be increased.

### Thirteenth Embodiment (Setting of Region-of-Interest Corresponding to Display Magnification)

The setting of the region-of-interest may be changed correspondingly to the display magnification of the operative field image captured by the surgical camera 11.

For example, the region-of-interest setting unit 81 sets the region-of-interest to a narrower region in a case where the operative field image is enlarged and displayed on the display 13, and sets the region-of-interest to a wider region in a case where the operative field image is reduced and displayed on the display 13.

With this arrangement, the region-of-interest having a size corresponding to the range displayed in the entire operative field image can be set.

### <Others>

Although the common region of the region-of-interest candidate and the segmentation region is set as the region-of-interest, the common region may be set on the basis of another relationship different from the common region. For example, in a case where the distance between the region-of-interest candidate and the segmentation region is shorter than a threshold distance, the entire of the region-of-interest candidate and the segmentation region can be set as the region-of-interest.

In this manner, the region-of-interest may be set on the basis of various relationships including the positional relationship between the region-of-interest candidate and the segmentation region.

### About Program

The series of processing described above can be executed by hardware or software. In a case where the series of processing is executed by software, a program constituting the software is installed on a computer built into dedicated hardware or a general-purpose personal computer from a program recording medium, or the like.

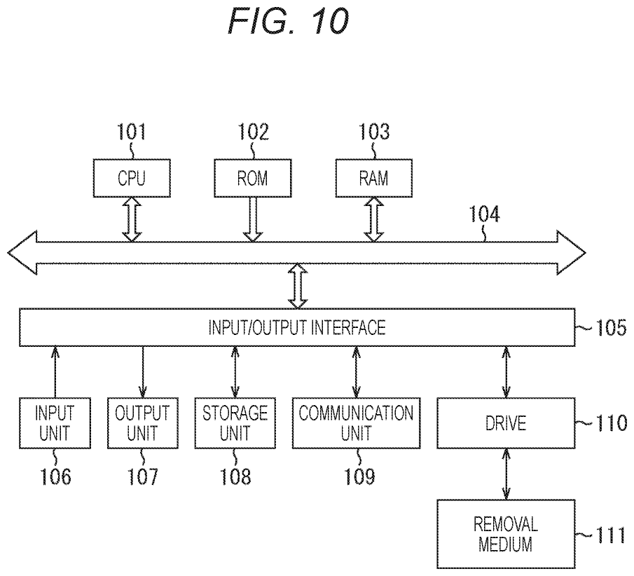

FIG. 10 is a block diagram illustrating a configuration example of hardware of a computer that executes the above-described series of processing by a program.

A central processing unit (CPU) 101, a read only memory (ROM) 102, and a random access memory (RAM) 103 are mutually connected by a bus 104.

The bus 104 is further connected with an input/output interface 105. An input unit 106, an output unit 107, a storage unit 108, a communication unit 109, and a drive 110 are connected to the input/output interface 105. The drive 110 drives a removable medium 111 such as a magnetic disk, an optical disk, a magneto-optical disk, or a semiconductor memory.

In the computer configured as described above, for example, the CPU 101 loads a program stored in the storage unit 108 into the RAM 103 via the input/output interface 105 and the bus 104 and executes the program to cause the above-described series of processing to be performed.

The program to be executed by the CPU 101 is provided, for example, by being recorded on the removable medium 111 or via a wired or wireless transmission medium such as a local area network, the Internet, or digital broadcasting, and is installed in the storage unit 108.

Note that the program executed by the computer may be a program in which processing is performed in time series in the order described in the present description, or may be a program in which processing is performed in parallel or at necessary timing such as when a call is made.

Note that the effects described in the present description are merely examples and are not restrictive, and there may be other effects.

Embodiments of the present technology are not limited to the above-described embodiments, and various modifications may be made without departing from the gist of the present technology.

For example, the present technology may be configured as cloud computing in which one function is shared by a plurality of devices over the network to perform the processing together.

Furthermore, each of the steps in the flowcharts described above can be executed by one device or executed by a plurality of devices in a shared manner.

Moreover, in a case where a plurality of parts of processing is included in one step, the plurality of parts of processing included in one step can be executed by one device or by a plurality of devices in a shared manner.

Note that, in the present description, a system means an assembly of a plurality of constituents (devices, modules (components), and the like), and it does not matter whether or not all the constituents are located in the same housing. Therefore, a plurality of devices stored in different housings and connected via a network and one device in which a plurality of modules is stored in one housing are both systems.

### Examples of Combinations of Configurations

The present technology can also have the following configurations.

(1)

A surgical system including:
an image processing unit that performs segmentation of an image captured by a camera and sets a segmentation region in which each target appears;
a region-of-interest candidate acquisition unit that acquires a region-of-interest candidate that is a region to be a candidate for a region-of-interest of an operator; and a control unit that sets the region-of-interest on the basis of a relationship between the segmentation region and the region-of-interest candidate.
   (2)

The surgical system according to (1), in which
the control unit sets a common region between the segmentation region and the region-of-interest candidate as the region-of-interest.
   (3)

The surgical system according to (1) or (2), in which
the control unit controls at least one of focus and exposure of the camera.
   (4)

The surgical system according to any one of (1) to (3), in which
the region-of-interest candidate acquisition unit acquires the region-of-interest candidate on the basis of input of at least one of a voice, a line-of-sight, a touch, a gesture, and a foot switch operation of the operator.
   (5)

The surgical system according to any one of (1) to (4), in which
the control unit sets the region-of-interest by using a plurality of the segmentation regions.
   (6)

The surgical system according to any one of (1) to (5), in which
the image processing unit sets the segmentation region in a region in which an operation target organ is displayed, the operation target organ being specified on the basis of surgical procedure information.
   (7)

The surgical system according to (6), in which
the control unit determines a surgical process on the basis of a positional relationship between a surgical tool and the operation target organ, and sets the region-of-interest on the basis of a determination result.
   (8)

The surgical system according to (6) or (7), in which
the image processing unit sets, on the basis of the surgical procedure information, a degree of importance in each portion of the segmentation region in which the operation target organ is displayed, and
the control unit sets the region-of-interest so as to include a portion where the degree of importance is higher than a threshold value.
   (9)

The surgical system according to any one of (1) to (8), in which
the image processing unit performs Depth estimation on the basis of the image captured by the camera, and on the basis of Depth information representing a result of the Depth estimation, performs division of the segmentation region or coupling of a plurality of the segmentation regions.
   (10)

The surgical system according to any one of (1) to (8), in which
the image processing unit performs SLAM processing on the basis of the image captured by the camera, and on the basis of SLAM information representing a result of the SLAM processing, performs division of the segmentation region or coupling of a plurality of the segmentation regions.
   (11)

The surgical system according to any one of (1) to (3), in which
the image processing unit presents information regarding the region-of-interest to the operator at the time of controlling the camera.
   (12)

The surgical system according to (11), in which
the control unit changes the region-of-interest in response to speech of the operator made after the information regarding the region-of-interest is presented.
   (13)

The surgical system according to any one of (6) to (8), further including
a surgical procedure information acquisition unit that acquires the surgical procedure information on the basis of at least one of information transmitted from a cooperative HIS and a recognition result of speech made before start of surgery.
   (14)

The surgical system according to any one of (1) to (13), in which
the control unit changes the region-of-interest correspondingly to display magnification of the image captured by the camera.
   (15)

A control method including, by a surgical system:
performing segmentation of an image captured by a camera and setting a segmentation region in which each target is displayed;
acquiring a region-of-interest candidate that is a region to be a candidate for a region-of-interest of an operator; and
setting the region-of-interest on the basis of a relationship between the segmentation region and the region-of-interest candidate.
   (16)

A program configured to cause a computer to execute processing of:
performing segmentation of an image captured by a camera and setting a segmentation region in which each target is displayed;
acquiring a region-of-interest candidate that is a region to be a candidate for a region-of-interest of an operator; and
setting the region-of-interest on the basis of a relationship between the segmentation region and the region-of-interest candidate.

### REFERENCE SIGNS LIST

1 Control device
2 Surgical procedure information providing device
11 Surgical camera
31 Region-of-interest candidate acquisition unit
32 Image processing unit
33 Control unit
34 Surgical procedure information acquisition unit
35 Segmentation target providing unit
36 Region-of-interest correction information acquisition unit
61 Segmentation processing unit
62 Region-of-interest superimposition processing unit
71 Segmentation weighting processing unit
72 Depth processing unit
73 SLAM processing unit
81 Region-of-interest setting unit

1. A surgical system comprising:
an image processing unit that performs segmentation of an image captured by a camera and sets a segmentation region in which each target appears;
a region-of-interest candidate acquisition unit that acquires a region-of-interest candidate that is a region to be a candidate for a region-of-interest of an operator; and
a control unit that sets the region-of-interest on a basis of a relationship between the segmentation region and the region-of-interest candidate.

2. The surgical system according to claim 1, wherein
the control unit sets a common region between the segmentation region and the region-of-interest candidate as the region-of-interest.

3. The surgical system according to claim 1, wherein
the control unit controls at least one of focus and exposure of the camera.

4. The surgical system according to claim 1, wherein
the region-of-interest candidate acquisition unit acquires the region-of-interest candidate on a basis of input of at least one of a voice, a line-of-sight, a touch, a gesture, and a foot switch operation of the operator.

5. The surgical system according to claim 1, wherein
the control unit sets the region-of-interest by using a plurality of the segmentation regions.

6. The surgical system according to claim 1, wherein
the image processing unit sets the segmentation region in a region in which an operation target organ is displayed, the operation target organ being specified on a basis of surgical procedure information.

7. The surgical system according to claim 6, wherein
the control unit determines a surgical process on a basis of a positional relationship between a surgical tool and the operation target organ, and sets the region-of-interest on a basis of a determination result.

8. The surgical system according to claim 6, wherein
the image processing unit sets, on a basis of the surgical procedure information, a degree of importance in each portion of the segmentation region in which the operation target organ is displayed, and
the control unit sets the region-of-interest so as to include a portion where the degree of importance is higher than a threshold value.

9. The surgical system according to claim 1, wherein
the image processing unit performs Depth estimation on a basis of the image captured by the camera, and on a basis of Depth information representing a result of the Depth estimation, performs division of the segmentation region or coupling of a plurality of the segmentation regions.

10. The surgical system according to claim 1, wherein
the image processing unit performs SLAM processing on a basis of the image captured by the camera, and on a basis of SLAM information representing a result of the SLAM processing, performs division of the segmentation region or coupling of a plurality of the segmentation regions.

11. The surgical system according to claim 3, wherein
the image processing unit presents information regarding the region-of-interest to the operator at a time of controlling the camera.

12. The surgical system according to claim 11, wherein
the control unit changes the region-of-interest in response to speech of the operator made after the information regarding the region-of-interest is presented.

13. The surgical system according to claim 6, further comprising
a surgical procedure information acquisition unit that acquires the surgical procedure information on a basis of at least one of information transmitted from a cooperative hospital information system (HIS) and a recognition result of speech made before start of surgery.

14. The surgical system according to claim 1, wherein
the control unit changes the region-of-interest correspondingly to display magnification of the image captured by the camera.

15. A control method comprising, by a surgical system:
performing segmentation of an image captured by a camera and setting a segmentation region in which each target is displayed;
acquiring a region-of-interest candidate that is a region to be a candidate for a region-of-interest of an operator; and
setting the region-of-interest on a basis of a relationship between the segmentation region and the region-of-interest candidate.

16. A program configured to cause a computer to execute processing of:
performing segmentation of an image captured by a camera and setting a segmentation region in which each target is displayed;
acquiring a region-of-interest candidate that is a region to be a candidate for a region-of-interest of an operator; and
setting the region-of-interest on a basis of a relationship between the segmentation region and the region-of-interest candidate.
